(19) United States
(12) Patent Application Publication
(10) Pub. No.: US 2008/0292567 A1
(43) Pub. Date: Nov. 27, 2008

(54) Uv-Protective Cosmetic Preparation and Use of Decorative Pigments Comprising a Protective Layer

(76) Inventors: Thomas Schuster, Lauf (DE); Peter Kruger, Lauf (DE); Ulrich Schmidt, Hersbruck (DE)

Correspondence Address: OSTROLENK FABER GERB & SOFFEN, 1180 AVENUE OF THE AMERICAS, NEW YORK, NY, 100368403, US

(21) Appl. No.: 10/599,097

(22) PCT Filed: Mar. 11, 2005
(86) PCT No.: PCT/EP2005/002615

§ 371 Date: Aug. 14, 2008

(30) Foreign Application Priority Data
Mar. 19, 2004 (DE) 10 2004 014 020.0

(51) Int. Cl. A61K 8/37; A61Q 17/04

(52) U.S. Cl. 424/59

(57) ABSTRACT

The invention relates to a UV-protective cosmetic preparation comprising one or more UV absorbers, which cosmetic preparation comprises effect pigments provided with at least one protective coating. The invention also relates to the use of effect pigments having at least one protective coating for the production of a UV protective cosmetic preparation.

The invention relates to a UV-protective cosmetic preparation and to the use of effect pigments having at least one protective coating for the production of a UV-protective cosmetic preparation.

Cosmetic preparations such as sunscreen agents are widely used in everyday life. A large number of sunscreen agents are marketed, particularly with a view to the damaging influences of UV light on the human skin which have become known in recent years. Development is proceeding to increasingly higher light protection factors in order to fulfill the need for reliable protection despite long periods of exposure to sunlight.

Damaging UV light is divided into the UV-A (320 to 400 nm) and UV-B (280 to 320 nm) ranges. On account of its shorter wavelengths, UV-B light is richer energy light. Excessive exposure of unprotected human skin to UV light can lead to skin cancer.

Benzophenones or avobenzone, for example, act as UV-A absorbers. Typical UV-B absorbers are p-aminobenzoic acid or cinnamates. Camphor derivatives, depending on their substitution, can have a wideband action in the entire UV range.

Sunscreen agents can contain pigments in addition to UV absorbers. These can act as a physical UV protection by covering the skin, i.e. by screening off the UV light.

DE 25 44 190 discloses a sunscreen agent which contains 4-isopropyl-dibenzoylmethane as a UV absorber.

CH 11639/78 and U.S. Pat. No. 4,387,089 disclose the use of 4-(1,1-dimethyl-ethyl)-4′-methoxydibenzoylmethane as a UV-A absorber which is reported to have a favorable effect on the action of UV-B absorbers.

In DE 33 02 123, 2,4-dimethyl-4′-methoxydibenzoylmethane and a sunscreen agent which contains this substance are described.

The subject of U.S. Pat. No. 6,210,658 is a sunscreen agent which contains UV-A and UV-B absorbers, and a barium compound for stabilization.

DE 41 23 772 and EP 1 078 883 B1 disclose the use of pyrogenically prepared titanium dioxide in sunscreen agents.

EP 1 078 957 B1 discloses the use of surface-modified, pyrogenically prepared titanium dioxide in sunscreen agents.

When using titanium dioxide particles in sunscreen agents, there is the disadvantage that after application of the sunscreen agent to the skin a white surface film is often produced—an effect which is not very attractive.

It might be conceivable to add pearlescent pigments to a sunscreen agent in order to produce a decorative effect on the skin after application of the sunscreen agent.

It has been found, however, that under the action of sunlight a breakdown of functional organic constituents, in particular a breakdown of the UV absorbers, can occur with effect pigments, in particular with pearlescent pigments, which is extremely undesirable, since this destroys the UV protection. Due to this destruction of UV protection, it is then necessary to re-apply the cosmetic preparation, for example a sunscreen agent, to the skin at relatively short time intervals in order to prevent damage to the skin by UV light.

There is, in particular with fashion-conscious consumers, the desire for cosmetics which on the one hand provide the skin with long-lasting UV protection and on the other hand fulfill a decorative purpose.

The object underlying the invention is achieved by providing a cosmetic preparation affording UV protection, comprising one or more UV absorbers and further comprising effect pigments provided with at least one protective coating.

Preferred developments are specified in subclaims 2 to 10.

The object underlying the invention is furthermore achieved by the use of effect pigments having at least one protective coating for the production of a UV-protective cosmetic preparation.

Preferred developments are specified in subclaims 12 to 18.

For the purposes of the invention, the term “effect pigments” is taken to mean a plurality of effect pigments which can have the same or different layer build-up. That is to say, the term “effect pigments” is also understood as meaning mixtures of various effect pigments. By mixing effect pigments having different optical characteristics, it is possible to produce specific color effects.

It has been found, surprisingly, that effect pigments, in particular pearlescent pigments, can be stabilized by the application of at least one protective coating such that no, or only an insignificant, degradation of functional organic constituents such as UV absorbers takes place in a cosmetic preparation subjected to sunlight radiation.

The cosmetic preparation according to the invention providing UV protection, which additionally contains effect pigments, preferably pearlescent pigments, having at least one protective coating, makes it possible not only to provide long-lasting UV protection but also to produce a variety of color effects on the skin. The effect pigments, preferably pearlescent pigments, having at least one protective coating align substantially parallel to the skin surface on account of their laminar structure. In particular when using pearlescent pigments having a protective coating, interesting color effects can be produced on the skin depending on the layer construction. In the case of pearlescent pigments, as a rule a layer or a number of layers of metal and/or metal oxides are applied to mica particles for the production of the color effects by interference.

Depending on the angle of incidence of the light and the layer build-up of the pigments, the skin then appears to have a different color or color shade. These luster and color effects impart an interesting appearance to the skin. In addition, the use of the cosmetic preparation according to the invention makes it possible to cover over unsightly areas, such as, for example, pigmentation disorders in the skin, parts of the skin affected by acne, scars, etc., which consequently has a beautifying effect.

Preferably, the at least one protective coating is substantially transparent. The—preferably substantially transparent—protective coating surrounds the effect pigments substantially completely, preferably completely.

Preferably, the protective coating is an inorganic protective coating. Preferably, a protective coating of SiO₂ is applied. The covering of effect pigments, preferably pearlescent pigments, with an SiO₂ layer can be simply carried out, for example by treatment of the effect pigments with water glass. A protective coating of SiO₂ is substantially transparent. In this regard, a protective coating of SiO₂ does not influence, or only insignificantly influences, the color effect produced by further metal and/or metal oxide layers applied to the pigment.

Of course, other or additional organic protective coatings, for example metal oxide layers, can be applied. Such a protective coating should be substantially chemically inert and cause isolation of the effect pigments, preferably pearlescent pigments, from the environment. The term “environment” is to be understood here as meaning the cosmetic preparation containing the functional organic constituents, for example the UV absorbers, into which the effect pigments are blended.

The present invention consequently makes it possible to provide cosmetic preparations or compositions containing a UV absorber or a number of UV absorbers, which additionally contain effect pigments which are coated by at least one protective coating and which are stable for a considerable period of time when exposed to, or irradiated by, sunlight. That is to say, the use of effect pigments provided with at least one substantially transparent protective coating in a cosmetic preparation affording UV protection causes no noticeable breakdown of the organic UV absorber or of the organic UV absorbers.

It has been found, surprisingly, that even effect pigments such as pearlescent pigments which have a TiO₂ coating stabilized by at least one protective coating, preferably an inorganic protective coating, are stabilized such that even the TiO₂ induces no degradation of organic compounds, such as, for example, organic UV absorbers.

By means of the preferably inorganic protective coating, the TiO₂ or the TiO₂ layer is isolated from the environment, such that no breakdown of constituents of the cosmetic preparation, such as, for example, UV absorbers, takes place. A very suitable protective coating has been found to be a protective coating of SiO₂.

The cosmetic preparation according to the invention, preferably a sunscreen agent, contains one or more organic UV absorbers in a suitable and conventional vehicle which is known to the person skilled in the art.

The UV absorber is preferably selected from the group which consists of benzophenones, hydroxynaphthoquinones, phenylbenz-oxazoles, phenylbenzimidazoles, digalloyl trioleate, aminobenzoic acid esters, salicylic acid esters, acyclic dienones, cinnamic acid esters, benzalazine, avobenzone, paraaminobenzoic acid and its derivatives, cinnamates, salicylates, camphor derivatives, benzimidazoles, 4-isopropyl-dibenzoylmethane, 4-(1,1-dimethylethyl)-4′-methoxydibenzoylmethane, 2,4-dimethyl-4′-methoxydibenzoylmethane and mixtures thereof.

The effect pigment is preferably a pearlescent pigment or a pigment having a layer-on-substrate build-up.

According to a preferred embodiment, the effect pigment contains titanium dioxide. Preferably, the effect pigment, preferably pearlescent pigment, has a layer construction in which, directly or indirectly, at least one protective coating of silicon dioxide is applied over the TiO₂ layer.

The protective coating of the effect pigment is preferably applied in an aqueous system, for example by the use of water glass.

The cosmetic preparation or composition can be present in the form of a cream, lotion, milk, emulsion, spray emulsion, jelly, oil, spray oil, or aerosol. Preferably, the cosmetic preparation is a sunscreen agent.

The examples below serve for the further illustration of the invention. The invention, however, is not restricted to these examples.

### EXAMPLE 1

### Coating of a Pearlescent Pigment

A solution of soda water glass 37/40 BE (11 g of soda water glass in 15 g of water) is introduced completely at 75° C. over a period of 15 min into a 10% strength suspension of pearlescent pigment (Prestige Silver supplied by ECKART, Fürth, Germany); the pH is not checked during this operation. On completion of the addition, the pH is lowered to pH 7.5 using dilute hydrochloric acid. The suspension is subsequently stirred at constant pH for 1 h, filtered off through a Buchner funnel, washed with 1000 ml of water and dried overnight at 120° C. in a drying oven.

### EXAMPLE 2

### Coating of a Pearlescent Pigment

A solution of soda water glass 37/40 BE (11 g of soda water glass in 15 g of water) is introduced completely at 75° C. over a period of 15 min into a 10% strength suspension of pearlescent pigment (Prestige Silver Star supplied by ECKART, Fürth, Germany); the pH is not checked during this operation. On completion of the addition, the pH is lowered to pH 7.5 using dilute hydrochloric acid. The suspension is subsequently stirred at constant pH for 1 h, filtered off through a Buchner funnel, washed with 1000 ml of water and dried overnight at 120° C. in a drying oven.

### EXAMPLE 3

### Preparation of a Moisturizing Cream Containing Pearlescent Pigments

Phase 1 and phase 2, which had a composition as indicated in Table 1 and Table 2 respectively, were in each case warmed to 78° C. Thereafter, phase 2 was added to phase 1 with homogenization. Subsequently, the mixture was allowed to cool to room temperature with stirring with to produce a cream.

In a first batch, 19.4 g of this cream were then blended with 0.6 g of Prestige Silver Star pigments, which had a coating of 2.5% by weight of SiO₂.

In conformity with the first batch, a second batch was prepared in which 19.4 g of the cream were likewise blended under identical conditions with 0.6 g of Prestige Silver Star pigments which, however, were not coated with SiO₂.

Finally, both batches were exposed to sunlight under identical conditions. Compared with a cream containing Prestige Silver Star without an SiO₂ coating, the degradation of the avobenzone was found to be greatly decreased.

TABLE 1
Composition of Phase I
Phase I:

| | Product | | |
|---|---|---|---|
| INCI name | description | % by wt | Supplier |
| Cyclomethicone | Dow Corning | 17.86% | Dow |
| | 345 fluid | | Corning |
| Dimethicone | Dow Corning | 4.96% | Dow |
| | 200 fluid, | | Corning |
| | 350 CST | | |
| C12-C15 alkyl benzoate | Tixogel FTN | 9.92% | Süd- |
| | | | Chemie |
| (and) stearalkonium bentonite | | | Rheo- |
| | | | logicals |
| (and) propylene carbonate | | | |
| Cetyldimethicone copolyol | Abil EM 90 | 1.19% | Degussa |
| Butylmethoxydibenzoylmethane | Parsol 1789 | 2.48% | Roche |
| (avobenzone) | | | |

TABLE 2
Composition of Phase II
Phase II:

| INCI name | Product description | % by wt | Supplier |
|---|---|---|---|
| Deionized water | | 62.62% | |
| Sodium chloride | | 0.37% | |
| Phenoxyethanol | Uniphen P-23 | 0.60% | Lipo Chemical |
| (and) methylparaben | | | |
| (and) butylparaben | | | |
| (and) propylparaben | | | |

1. A UV-protective cosmetic preparation comprising one or more UV absorbers,
characterized in that
said cosmetic preparation contains effect pigments, which effect pigments have at least one protective coating.

2. A cosmetic preparation as defined in claim 1,
characterized in that
said protective coating is substantially transparent.

3. A cosmetic preparation as defined in claim 1 or claim 2,
characterized in that
said at least one protective coating is an inorganic protective coating.

4. A cosmetic preparation as defined in any one of the previous claims,
characterized in that
said UV absorber is an organic UV absorber, which is preferably selected from the group consisting of benzophenones, hydroxynaphthoquinones, phenylbenzoxazoles, phenylbenzimidazoles, digalloyl trioleate, aminobenzoic acid esters, salicylic acid esters, acyclic dienones, cinammic esters, benzalazine, avobenzone, paraaminobenzoic acid and derivatives thereof, cinnamates, salicylates, camphor derivatives, benzimidazoles, 4-isopropyldibenzoylmethane, 4-(1,1-dimethylethyl)-4′-methoxydibenzoylmethane, 2,4-dimethyl-4′-methoxydibenzoylmethane and mixtures thereof.

5. A cosmetic preparation as defined in any one of the previous claims,
characterized in that
said effect pigments have a layer-on-substrate build-up.

6. A cosmetic preparation as defined in any one of the previous claims,
characterized in that
said effect pigments are pearlescent pigments.

7. A cosmetic preparation as defined in any one of the previous claims,
characterized in that
said effect pigments contain titanium(IV) oxide.

8. A cosmetic preparation as defined in any one of the previous claims,
characterized in that
said effect pigments exhibit at least one protective coating of silicon dioxide.

9. A cosmetic preparation as defined in any one of claims 1 to 8,
characterized in that
it exists in the form of a cream, lotion, milk, emulsion, spray emulsion, jelly, oil, spray oil, or aerosol.

10. A cosmetic preparation as defined in any one of the previous claims,
characterized in that
said cosmetic preparation is a sunscreen.

11. Use of effect pigments for the production of a UV-protective cosmetic preparation, wherein the effect pigments have at least one protective coating.

12. Use as defined in claim 11,
characterized in that
said protective coating is substantially transparent.

13. Use as defined in claim 11 or claim 12,
characterized in that
said at least one protective coating is an inorganic protective coating.

14. Use as defined in any one of claims 11 to 13,
characterized in that
said effect pigments have a layer-on-substrate build-up.

15. Use as defined in any one of claims 11 to 14,
characterized in that
said effect pigments are pearlescent pigments.

16. Use as defined in any one of claims 11 to 15,
characterized in that
said effect pigments contain titanium(IV) oxide.

17. Use as defined in any one of claims 11 to 16,
characterized in that
said effect pigments exhibit at least one protective coating of silicon dioxide.

18. Use as defined in any one of claims 11 to 17,
characterized in that
said cosmetic preparation is a sunscreen.
